(19) United States
(12) Patent Application Publication
(10) Pub. No.: US 2008/0003280 A1
(43) Pub. Date: Jan. 3, 2008

(54) Combination cough treatment compounds and method of treating common coughs

(76) Inventors: Brian M. Levine, Coto de Caza, CA (US); William Berger, Coto de Caza, CA (US)

Correspondence Address: STETINA BRUNDA GARRED & BRUCKER, 75 ENTERPRISE, SUITE 250, ALISO VIEJO, CA, 92656, US

(21) Appl. No.: 11/820,545

(22) Filed: Jun. 20, 2007

Related U.S. Application Data
(60) Provisional application No. 60/805,860, filed on Jun. 26, 2006.

(51) Int. Cl. A61K 9/48; A61K 31/495; A61K 31/522; A61P 43/00; A61K 9/28

(52) U.S. Cl. 424/456; 424/463; 424/474; 514/255.04; 514/263.34

(57) ABSTRACT

A novel composition of three recognized antitussive agents, when used in combination, work in an additive fashion to suppress cough. Each drug has a desirable effect of suppressing cough in a unique fashion. However, undesirable side effects can occur in humans at concentrations at which the drug has its maximal antitussive effect. Pharmaceutical compositions of theobromine, dextromethorphan, and an antihistamine with central nervous system effect, such as dexbrompheniramine, maximize cough suppression while decreasing the likelihood of side effects when used in combination.

### CROSS-REFERENCE TO RELATED APPLICATIONS

This application claims the benefit of U.S. Provisional Application No. 60/805,860, filed on Jun. 26, 2006, the teachings of which are expressly incorporated by reference.

STATEMENT RE: FEDERALLY SPONSORED RESEARCH/DEVELOPMENT

Not Applicable

### BACKGROUND

Cough is the most common symptom for which patients seek medical attention in an outpatient setting in the United States. Cough, although a common symptom, may have one or a combination of causes. For example, cough may be a result of a simple viral upper respiratory infection, of short duration, lasting but a few weeks (acute cough). However, cough can be persistent, lasting for several weeks, months, or even years (chronic cough). Chronic cough may be caused by continuous mucus drainage down the throat, asthma, gastroesophageal reflux, a variety of pulmonary disorders, and even as a side effect of certain medications. In some cases, coughing serves as a protective mechanism by preventing aspiration of foreign material into the lungs or, as with infectious processes, expulsion of unwanted mucus and pathogens from the airway. However, in many cases of chronic cough, the mechanism serves no useful purpose and may dramatically affect one's entire lifestyle causing sleeplessness, exhaustion, annoyance, self consciousness, and social limitation. Physical consequences may be hoarseness, incontinence of urine or stool, perspiration, and chest wall pain. Therefore, in those situations where cough serves no useful purpose, the benefit of medicines to suppress cough, termed antitussives, are highly desirable. The coughing mechanism is a reflex arc that is initiated by stimulation of specialized sensory nerve fibers, called receptors, distributed throughout the respiratory tract with greatest concentration in the upper airways. There are actually several different receptor types that respond to chemical and mechanical stimuli. After receptor stimulation, impulses travel away along nerves (afferent limb), to intermediate nerve terminal sites (ganglions), where connecting nerves intersect to further transmit impulses to the cough center in the brain (medulla). At the central command center in the brain, all nerve impulses are integrated, and a coordinated set of nerve impulses are generated to nerves (efferent limb) leading to the expiratory muscles that contract to produce an effective cough. Medicines that have an antitussive effect may work at one or at a combination of sites along the reflex arc. Since there are many sites in the cough reflex arc that, when stimulated, can initiate a response, then it is reasonable to discern that antitussive agents, working at different sites in combination have a greater likelihood of treatment success. Furthermore, since all medications have toxic potential at high doses, it stands to reason that a synergistic combination can provide an adequate response at lower drug concentrations, hence minimizing untoward side effects.

The American College of Chest Physicians in 2006 issued guidelines for cough evaluation and management. A conclusion of their guidelines was that “most over the counter cough medications are ineffective”. The present invention is unprecedented in that there is now available a pharmaceutical composition of clinically proven antitussive agents, working in concert, specifically indicated for the treatment of cough.

### BRIEF SUMMARY

One embodiment of the present invention contemplates an antitussive pharmaceutical composition comprising, in operative combination, theobromine; dextromethorphan; and an antihistamine. The antihistamine may consist of dexbrompheniramine, dexchlorpheniramine, brompheniramine, chlorpheniramine, diphenhydramine, cetirizine, azatadine, clemestine, doxylamine, pyrilamine, triprolidine, tripelennamine, cyproheptadine, carbinoxamine, bromodiphenhydramine, phenindamine, or pharmaceutically acceptable salts of any the antihistamines.

The antitussive pharmaceutical composition may include at least 10 mg of theobromine or a pharmaceutically acceptable salt thereof. More particularly, the antitussive may include from 10 mg to 3000 mg of theobromine or a pharmaceutically acceptable salt thereof.

Additionally, or alternatively, the antitussive pharmaceutical composition may include at least 2 mg of dextromethorphan or a pharmaceutically acceptable salt thereof. And more particularly, may include from 2 mg to 60 mg of dextromethorphan or a pharmaceutically acceptable salt thereof.

The antitussive pharmaceutical composition may include at least 1 mg of an antihistamine or a pharmaceutically acceptable salt thereof. And more particularly, may include from 1 mg to 100 mg of the antihistamine or a pharmaceutically acceptable salt thereof.

The antitussive pharmaceutical composition may optionally further include a pharmaceutically acceptable non-toxic carrier. The antitussive composition may also be in the form of a single formulation. The formulation may take the form of a liquid, a pill, a tablet, or a capsule. If the antitussive composition is in the form of a tablet, it may be a chewable tablet, a melting tablet, or an enteric-coated tablet. If the antitussive composition is in the form of a capsule, it may be a liquid gelatin capsule or an enteric-coated capsule.

The antitussive pharmaceutical composition may be adapted to prevent release of the antitussive agents or pharmaceutically acceptable salt in the stomach. The antitussive composition may be a slow-release formulation adapted to release the antitussive agents or pharmaceutically acceptable salt thereof over a period of time. Alternatively, the antitussive composition may be a transdermal formulation adapted to release the antitussive agents or pharmaceutically acceptable salt thereof over a period of time.

Also contemplated is a method for the treatment of cough in an individual, wherein the individual is administered the antitussive composition of the present invention. Another contemplated method of the present invention includes a therapeutic antitussive effect in an individual achieved by intravenously administering theobromine to the individual. Yet another embodiment of the invention contemplates achieving a therapeutic antitussive effect in an individual by administering at least two antitussive agents to the individual. The antitussive agents are theobromine, dextromethorphan, and an antihistamine. The antihistamine in this method may be dexbrompheniramine, dexchlorpheniramine, brompheniramine, chlorpheniramine, diphenhydramine, cetirizine, azatadine, clemestine, doxylamine, pyrilamine, triprolidine, tripelennamine, cyproheptadine, carbinoxamine, bromodiphenhydramine, phenindamine, or a pharmaceutically acceptable salt of one of the antihistamines.

In this last method, all three antitussive agents may be administered to the individual. The antitussive agents may be in the form of a single formulation or may each be present in its own formulation. Regardless of whether this method utilized a single formulation or individual formulations, the form of each may be a liquid, a pill, a tablet, or a capsule.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is a structural view of theobromine;

FIG. 2 is a graph showing the antitussive effect of theobromine in comparison to codeine and caffeine;

FIG. 3 is a structural view of dextromethorphan;

FIG. 4 is a graph showing the antitussive effect of dextromethorphan in comparison to a placebo; and

FIG. 5 is a structural view of dexbrompheniramine.

### DETAILED DESCRIPTION

The detailed description set forth below is intended as a description of the presently preferred embodiment of the invention, and is not intended to represent the only form in which the present invention may be constructed or utilized. The description sets forth the functions and sequences of steps for constructing and operating the invention. It is to be understood, however, that the same or equivalent functions and sequences may be accomplished by different embodiments and that they are also intended to be encompassed within the scope of the invention.

The present invention relates to novel pharmaceutical compositions of three antitussive agents in varying concentrations, namely, theobromine, dextromethorphan, and an H1 receptor antagonist (antihistamine), preferably an “older” generation antihistamine, such as dexbrompheniramine.

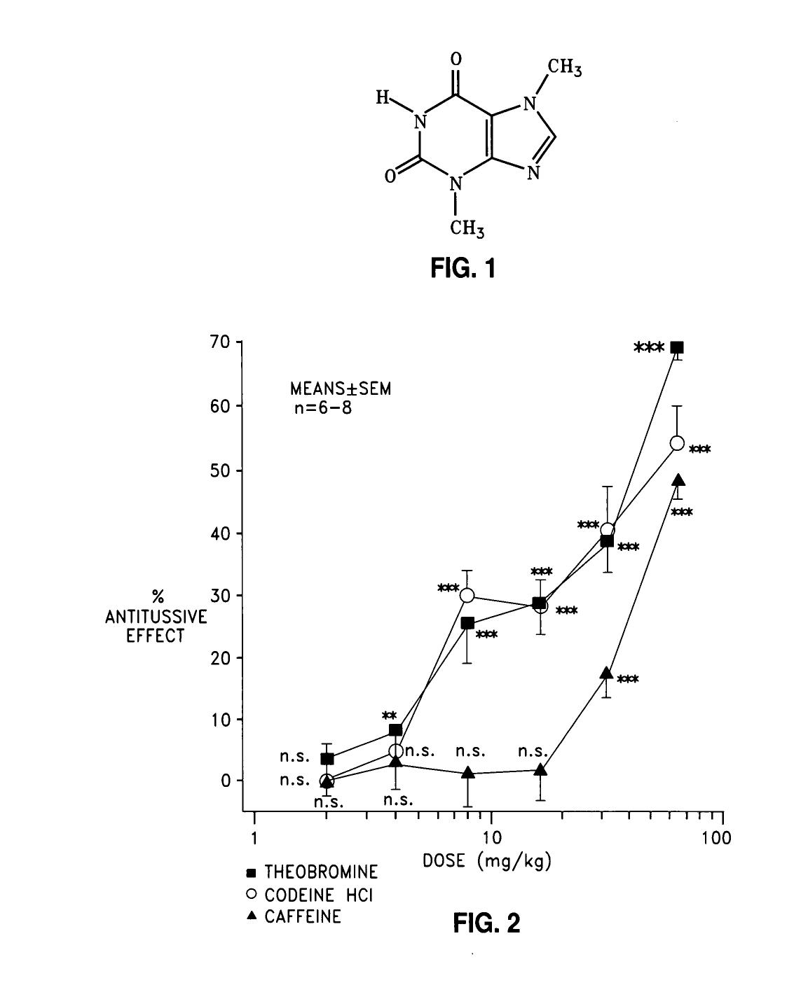

Theobromine [3,7-dihydro-3,7-dimethyl-1H-purine-2,6-dione], shown in FIG. 1, is a natural methylxanthine alkaloid found in leaves of tea, cocoa-seed and coffee-berry. Theobromine exhibits pharmacological actions of therapeutic interest, such as relaxation of smooth muscle, notably bronchial muscle, stimulation of the central nervous system (CNS), stimulation of cardiac muscle, and diuretic effect on the kidney. The risk of dangerous side effects caused by theobromine, however, is negligible compared to other methylxanthines such as theophyline or caffeine. Theobromine has recently been determined to have a strong and long lasting antitussive effect, comparable, in fact to codeine, as shown in FIG. 2. The antitussive effect was demonstrated to be as a result of suppression of vagus nerve sensory depolarization in patients whose cough was induced with a capsaicin challenge. Also, discovered in this study was that theobromine stimulates mucociliary clearance, has low toxicity and has few deleterious side effects, such as those exhibited by narcotic antitussive agents.

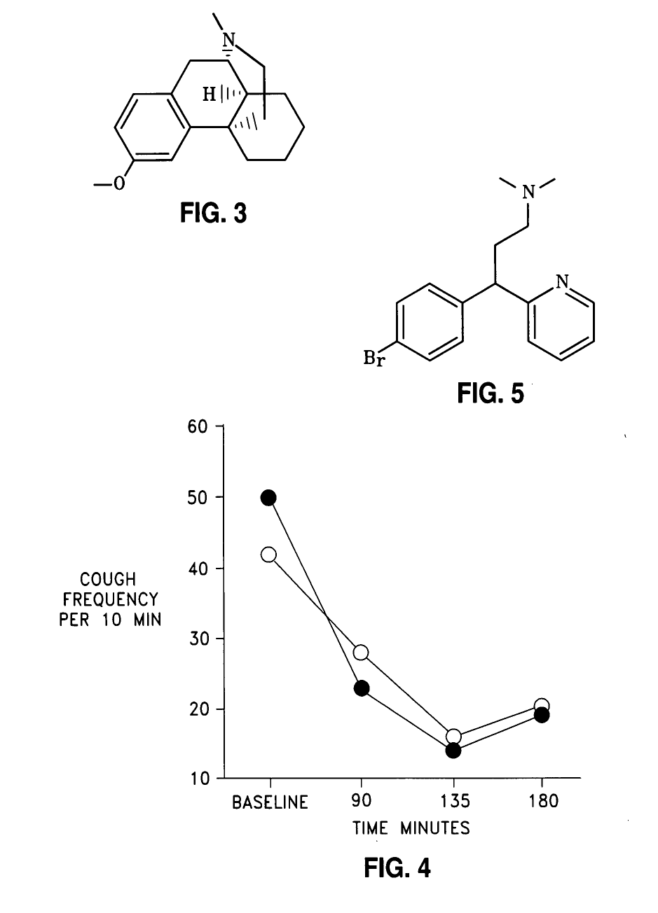

Dextromethorphan [C₁₈H₂₅NO], shown in FIG. 3, is a well known antitussive agent included in many over the counter cold/cough preparations. It is classified as on opioid analgesic and is the dextro isomer molecule of morphine. Dextromethorphan exhibits its pharmacological action by direct action on the cough center in the medulla area of the brain. It specifically acts as an antagonist to the NMDA glutamate receptor and the alpha 3/beta 4 nicotinic receptor, an agonist at the sigma 1 and sigma 2 opioid receptors, and affects the serotonin reuptake pump. Dextromethorphan is virtually devoid of opiate side effects but can cause occasional constipation, drowsiness, or euphoria. The most commonly recommended dosage in adults is 15-30 mg. However, in these dose ranges, the antitussive effect of dextromethorphan is no greater than placebo, as shown in FIG. 4 wherein the placebo is represented by unshaded circles and a 30 mg dose of dextromethorphan is represented by the shaded circles. Significant cough suppression does, in fact, occur at 60 mg/dose in adults. However, in that dose range the likelihood of side effects, such as euphoria becomes much greater. A synergistic drug combination, such as with theobromine and an antihistamine, would allow dextromethorphan to be more effective at lower doses without causing deleterious side effects. Furthermore, with antitussive success at lower doses, the likelihood of substance abuse is lessened.

Dexbrompheniramine [3-(4-bromophenyl)-N,N-dimethyl-3-pyridin-2-yl-propan-1 -amine], shown in FIG. 5, is an “older” generation antihistamine that competitively binds to histamine-H1-receptors throughout body tissues. The traditional use of antihistamines has proven effective in relieving allergic symptoms, such as sneezing, watery and itchy eyes, as well as rhinorhea. However, studies have indicated an improvement in cough in patients with an upper respiratory tract infection when an older antihistamine (brompheniramine) was used in combination with a decongestant. In contrast, studies have shown that newer generation antihistamines are relatively ineffective. It is a widely accepted fact that older antihistamines, such as dexbrompheniramine, have a sedative central nervous system effect, as well as a peripheral anticholinergic effect, in reducing cough. Furthermore, newer studies suggest that dexbrompheniramine affects the vanilloid VR 1 sensory peripheral airway receptors to reduce cough. Other older antihistamines that most likely have the same clinical effect include chlorphenirmine, dexchlorpheniramine, cetirizine, azatadine, clemestine, doxylamine, diphenhydramine, and others. The d-isomer isolated antihistamine provides a markedly enhanced antihistaminic effect while being substantially free of untoward side effects. Several commercially available preparations contain either an older antihistamine and/or dextromethorphan. However, these medications are usually combined with either a decongestant, an expectorant, analgesic, anti-inflammatory, or narcotic drug preparation. U.S. Pat. No. 6,979,689 (Compositions and methods for treating upper respiratory congestion), as well as U.S. Pat. No. 4,619,934 (Cough/cold mixtures comprising non-steroidal anti-inflammatory drugs), document such drug combinations. The drug combinations are designed to treat not only cough but also airway congestion and/or inflammation that may exist with an infectious or allergic malady. However, side effects such as respiratory depression, with the use of narcotics, or extreme airway dryness, with the use of a decongestant, produce unwanted, as well as unnecessary side effects in patients having a chronic cough. A preparation designed to treat only cough minimizes side effects and maximizes effectiveness.

Theobromine, used commercially for years in food preparations, has recently been found to have antitussive effect. To date, there is no published data to suggest that theobromine has been included in a synergistic, pharmaceutical composition that causes cough suppression. U.S. Pat. No. 6,348,470 (Antitussive compositions) describes the antitussive effect of theobromine and gives examples of delivery modality, but does not specify a more desirable effect of cough suppression when used with other cough suppressants, such as dextromethorphan and older antihistamines, such as dexbrompheniramine.

The pharmaceutical composition and delivery method of the three combined antitussive agents varies greatly depending on the causes of cough, the age and size of the patient, the desired degree and/or duration of the cough suppression, the sensitivity of response, and underlying medical problems that affect pharmacodynamics. The term “effective amount,” as used herein, is intended to refer to an amount effective for bringing about an improvement in the condition, or relief from the symptom of cough. Further, the effective amount refers to an amount in a single dose of the formulation.

An amount of dextromethorphan proven to be effective is generally within the range of 2 mg and 60 mg. Even though it has an excellent safety profile and devoid of opiate side effects, dextromethorphan at doses higher than 60 mg can cause constipation, drowsiness or euphoria. An amount of theobromine proven to be effective is generally within the range of 10-3000 mg. At higher doses, theobromine cannot only cause myocardial stimulation and vasodilation, but also relaxation of the lower esophageal sphincter, worsening acid reflux and possibly stimulating rather than relieving cough symptoms.

Most “older” antihistamines are effective at relieving cough. The existing older generation antihistamines and their recommended effective doses are listed in Table 1. The effective dose range is generally within 1 mg to 100 mg. Large doses can cause drowsiness and/or dizziness. Even though “older” antihistamines have been shown to be effective at reducing cough, any “newer” antihistamine may indeed prove to demonstrate some antitussive effect, when combined in a synergistic fashion with dextromethorphan and theobromine. Therefore, all antihistamines can be included in the novel composition with the desirable result being cough suppression.

TABLE 1

| DRUG (SALT FORM) | USUAL SINGLE ADULT DOSE | |
|---|---|---|
| chlorpheniramine (maleate) | 2-4 | mg |
| brompheniramine (maleate) | 8-12 | mg |
| dexchlorpheniramine (maleate) | 2-6 | mg |
| dexbrompheniramine (maleate) | 6 | mg |
| diphenhydramine (HCl) | 12.5-50 | mg |
| bromodiphenhydramine (HCl) | 3.75 | mg |
| azatadine (maleate) | 1-2 | mg |
| pyrilamine (maleate, tannate) | 12.5 | mg |
| doxylamine (succinate) | 7.5-10 | mg |
| carbinoxamine (maleate) | 4 | mg |
| tripelennamine (HCl) | 25-50 | mg |
| tripolidine (HCl) | 1.25-2.5 | mg |
| cetirizine (HCl) | 10 | mg |
| clemestine (fumarate) | 1.34-2.68 | mg |

Each of the three agents can be combined as a salt, i.e., maleate, stearate, succinate, tannate, tartrate, HCl, or HBr to become effective in the pharmaceutical composition. The foregoing active ingredients will typically be administered in admixture with suitable pharmaceutical diluents, excipients or carriers (collectively referred to herein as “carrier” materials) suitably selected with respect to the intended form of administration, i.e., oral tablets, capsules, elixirs, syrups, etc. and consistent with conventional pharmaceutical practices. For instance, for oral administration in the form of tablets or capsules, the active drug components may be combined with any oral non-toxic pharmaceutically acceptable inert carrier such as lactose, starch, sucrose, cellulose, magnesium stearate, dicalcium phosphate, calcium sulfate, mannitol, ethyl alcohol (liquid forms), and the like. Moreover, when desired or necessary, suitable binders, lubricants, disintegrating agents and coloring agents can also be incorporated in the mixture. Suitable binders include starch, gelatin, natural sugars, corn sweeteners, natural and synthetic gums such as acacia, sodium alginate, carboxymethylcellulose, polyethylene glycol and waxes. The lubricants that may be used include boric acid, sodium benzoate, sodium acetate, sodium chloride, etc. Disintegrators include, without limitation, starch, methylcellulose, agar, bentonite, guar gum, etc. Sweetening and flavoring agents and preservatives can also be included where appropriate. Alternatively, it is contemplated that at least two of the agents, or active ingredients, may be administered to the patient independently in order to achieve the desired synergistic effect. For example, an over-the-counter packaging may contain separate pills of two or more of the agents. The patient may then take each pill contemporaneously, rather than taking the combined agents in a single dosage.

It is further contemplated that the pharmaceutical compositions of the present invention, and in particular theobromine, may be administered intravenously in therapeutically effective amounts sufficient to induce an antitussive effect. Along these lines, it is expressly contemplated that the intravenous administration of theobromine by itself, may be provide a significant therapeutic benefit as an antitussive agent. As will be readily understood by those skilled in the art, such intravenous administration may involve either the bolus administration of theobromine or, alternatively, the theobromine may be administered in a particular amount per kilogram of body mass in either one or multiple doses per day, as may be appropriate to provide an antitussive affect for a particular individual. Accordingly, it should be expressly understood that the intravenous administration of the antitussive agents discussed herein, and in particular theobromine by itself, is considered to fall within the scope of the present invention.

Of course, additionally, the compositions of the present invention may be formulated in sustained release form to provide the rate controlled release of any one or more of the components to optimize the therapeutic effects, while minimizing undesirable side effects. Suitable dosage forms for sustained release include layered tablets containing layers of varying disintegration rates or controlled release polymeric matrices impregnated with the active components and shaped in tablet form or capsules containing such impregnated or encapsulated porous polymeric matrices.

As representative suitable formulations consistent with the objects, features and advantages of the present invention, the following non-limiting examples are provided. In the first example, an adult dose capsule was prepared containing 6 mg of dexbrompheniramine maleate, 30 mg of dextromethorphan-HBr, 350 mg of theobromine, and starch glycolate to the selected size. In a second example, an adult dose of a prolonged release suspension was prepared containing 8 mg of brompheniramine tannate, 20 mg of dextromethorphan tannate, 250 mg of theobromine, aroma, saccharose, color, preservant, methylcellulose, and water. Although the above formulations have been shown, it is contemplated that the medication may be prepared in a variety of forms, such as, but not limited to, ointments, creams, gels, transdermals, solutions, sprays, suspensions, tablets, caplets, pellets, patches, capsules, and any other sterile or non-sterile dosage delivery system either now known or created in the future.

A study comprising eight patients was conducted to evaluate the efficacy, dose range, and potential side effects of the medication of the present invention. Each of the eight patients had previously unsuccessfully been treated for chronic cough and had been diagnosed as having “unexplained” or “idiopathic” chronic cough with no known cause for the cough. Each patient had a persistant, unabated cough lasting for several years that profoundly diminished his or her quality of life that was previously untreatable other than with narcotic cough suppressants. All patients knowingly volunteered for the study and informed consent was obtained from each patient. The patients participated in a three week study that was preceded by an office evaluation to rule out any recent or previously unrecognized cause of cough. All previous drugs known to alleviate cough, including narcotic preparations, were eliminated from use during the study. The combination medication of the present invention was then prescribed on a weekly basis with an office evaluation at the end of each weekly regimen. At the end of the study, the medication was terminated and a follow up office visit was scheduled a week later. Finally, a telephone evaluation was made two weeks after drug termination. The dosage, of each of the three components of the medication, was adjusted on a weekly basis depending on drug efficacy and/or side effects. Success was based on diminished frequency and intensity of the cough as well as a determined improvement in quality of life by implementing the Leicester Cough Questionnaire. The results of such study are shown in Table 2. It is to be noted that evaluation of patient number 4 was terminated after one week due to severe sinus infection and that patients 2, 3, and 6 required narcotic cough suppressant prior to evaluation.

TABLE 2

| | | Days to | Benefit | Benefit at more |
|---|---|---|---|---|
| | Maximum | maximum | at end of | than 14 days |
| Patient Number | benefit | benefit | treatment | after treatment |
| 1 | 90% | 2 | 90% | 90% |
| 2 | 70% | 2 | 70% | 0% |
| 3 | 50% | 7 | 50% | 0% |
| 4 | 50% | 7 | 50% | 0% |
| 5 | 20% | — | 20% | 0% |
| 6 | 100% | 2 | 100% | 100% |
| 7 | 80% | 4 | 80% | — |
| 8 | 50% | 7 | 50% | — |

From the foregoing, other typical acceptable pharmaceutical formulations will be apparent to those skilled in the art of pharmaceutical formulations.

The above description is given by way of example, and not limitation. Given the above disclosure, one skilled in the art could devise variations that are within the scope and spirit of the invention disclosed herein, including utilizing effective dosages other than the preferred ranges set forth herein above with respect to the active ingredients may be applicable as a consequence of variations of the responsiveness of the patient treated, severity of symptoms, dosage related adverse effects, if any, observed and similar considerations. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. An antitussive pharmaceutical composition comprising, in operative combination, (i) theobromine; (ii) dextromethorphan; and (iii) an antihistamine.

2. The antitussive pharmaceutical composition of claim 1 wherein said antihistamine is selected from the group consisting of dexbrompheniramine, dexchlorpheniramine, brompheniramine, chlorpheniramine, diphenhydramine, cetirizine, azatadine, clemestine, doxylamine, pyrilamine, triprolidine, tripelennamine, cyproheptadine, carbinoxamine, bromodiphenhydramine, phenindamine, and pharmaceutically acceptable salts thereof.

3. The antitussive pharmaceutical composition of claim 1 comprising at least 10 mg of theobromine or a pharmaceutically acceptable salt thereof.

4. The antitussive pharmaceutical composition of claim 3 comprising from 10 mg to 3000 mg of theobromine or a pharmaceutically acceptable salt thereof.

5. The antitussive pharmaceutical composition of claim 1 comprising at least 2 mg of dextromethorphan or a pharmaceutically acceptable salt thereof.

6. The antitussive pharmaceutical composition of claim 5 comprising from 2 mg to 60 mg of dextromethorphan or a pharmaceutically acceptable salt thereof.

7. The antitussive pharmaceutical composition of claim 1 wherein said antihistamine consists of dexbrompheniramine or a pharmaceutically acceptable salt thereof.

8. The antitussive pharmaceutical composition of claim 1 comprising at least 1 mg of said antihistamine or a pharmaceutically acceptable salt thereof.

9. The antitussive pharmaceutical composition of claim 8 comprising from 1 mg to 100 mg of said antihistamine or a pharmaceutically acceptable salt thereof.

10. The antitussive pharmaceutical composition of claim 1 further comprising a pharmaceutically acceptable non-toxic carrier.

11. The antitussive pharmaceutical composition of claim 1 wherein the composition is in the form of a single formulation selected from the group consisting of a liquid, a pill, a tablet, and a capsule.

12. The antitussive pharmaceutical composition of claim 11 wherein the tablet is selected from the group consisting of a chewable tablet, a melting tablet, and an enteric-coated tablet.

13. The antitussive pharmaceutical composition of claim 11 wherein the capsule is selected from the group consisting of a liquid gelatin capsule and an enteric-coated capsule.

14. The antitussive pharmaceutical composition of claim 1 wherein the formulation is a liquid.

15. The antitussive pharmaceutical composition of claim 1 wherein the formulation is adapted to prevent release of the antitussive agents or pharmaceutically acceptable salt thereof in the stomach.

16. The antitussive pharmaceutical composition of claim 1 wherein the formulation is a slow-release formulation adapted to release the antitussive agents or pharmaceutically acceptable salt thereof over a period of time.

17. The antitussive pharmaceutical composition of claim 1 wherein the formulation is a transdermal formulation adapted to release the antitussive agents or pharmaceutically acceptable salt thereof over a period of time.

18. A method for the treatment of cough in an individual, said method comprising administering to such individual the antitussive pharmaceutical composition of claim 1.

19. A method for providing a therapeutic antitussive effect in an individual comprising the step:
a) intravenously administering theobromine to the individual.

20. A method for providing a therapeutic antitussive effect in an individual comprising the step:
a) administering at least two antitussive agents to the individual, said antitussive agents selected from the group consisting of theobromine, dextromethorphan, and an antihistamine.

21. The method of claim 20 wherein said antihistamine is selected from the group consisting of dexbrompheniramine, dexchlorpheniramine, brompheniramine, chlorpheniramine, diphenhydramine, cetirizine, azatadine, clemestine, doxylamine, pyrilamine, triprolidine, tripelennamine, cyproheptadine, carbinoxamine, bromodiphenhydramine, phenindamine, and pharmaceutically acceptable salts thereof.

22. The method of claim 20 wherein in step (a) all three antitussive agents are administered to the individual.

23. The method of claim 20 wherein the antitussive agents are in the form of a single formulation.

24. The method of claim 23 wherein the single formulation is selected from the group consisting of a liquid, a pill, a tablet, and a capsule.

25. The method of claim 20 wherein each antitussive agent is in its own formulation.

26. The method of claim 25 wherein each formulation is selected from the group consisting of a liquid, a pill, a tablet, and a capsule.
